(19) United States
(12) Patent Application Publication
(10) Pub. No.: US 2018/0256387 A1
(43) Pub. Date: Sep. 13, 2018

(54) A PILLOW

(71) Applicant: PATNEY LIMITED, Hamilton (NZ)

(72) Inventor: Frances Patricia ANDERSON, Hamilton (NZ)

(73) Assignee: PATNEY LIMITED, Hamilton (NZ)

(21) Appl. No.: 15/753,638

(22) PCT Filed: Aug. 25, 2016
(86) PCT No.: PCT/NZ2016/050136

§ 371 Date: Feb. 20, 2018

(30) Foreign Application Priority Data
Aug. 25, 2015 (NZ) 711399

(51) Int. Cl. A61F 5/56; A47G 9/10

(52) U.S. Cl. CPC A61F 5/56; A47G 2009/1018; A47G 9/1081

(57) ABSTRACT

The present invention provides a pillow or sleep positioner comprising a main body, the main body comprising an upper surface and a lower surface, and a neck supporting portion and head supporting portion, wherein the main body comprises a region of increased deformability relative to other parts of the main body such that when a user's head is resting on the head supporting portion, a recess, preferably a concave recess, defines a head receiving portion in an upper surface of the head supporting portion.

The present invention relates to pillows, and in particular, but not exclusively, to a pillow which reduces or eliminates snoring in the user.

### BACKGROUND

The prior art includes many examples of devices, and in particular pillows, which are said to reduce snoring.

Many anti-snore devices prevent the user sleeping on their back or encourage the user not to sleep on their back. However these are often ineffective in practice, as many people snore regardless of whether they sleep on their back or side.

A number of otherwise credible anti-snore devices are invasive and uncomfortable to use. Often the user decides that the benefit of reduced snoring is outweighed by the discomfort involved in using the device, and so does not use it consistently, if at all.

Still more of the anti-snore devices of the prior art simply do not work.

The reference to any prior art in the specification is not, and should not be taken as, an acknowledgement or any form of suggestion that the prior art forms part of the common general knowledge in any country.

### OBJECT OF THE INVENTION

It is an object of the invention to provide a pillow or sleep positioner which is relatively comfortable to use and which is effective at reducing snoring. It is an alternative object to provide a pillow or sleep positioner which assists the user in maintaining good sleep posture or positioning. It is a further alternative object to at least provide the public with a useful choice.

Other objects of the present invention may become apparent from the following description, which is given by way of example only.

### BRIEF SUMMARY OF THE INVENTION

According to one aspect of the present invention there is provided a pillow or sleep positioner comprising a main body, the main body comprising:

an upper surface and a lower surface; and

a neck supporting portion and head supporting portion, wherein:
the main body comprises a region of increased deformability relative to other parts of the main body such that when a user's head is resting on the head supporting portion, a recess, preferably a concave recess, defines a head receiving portion in an upper surface of the head supporting portion.

According to another aspect of the present invention there is provided a pillow or sleep positioner comprising a main body, the main body comprising:

an upper surface and a lower surface; and

a neck supporting portion and head supporting portion, wherein:
the main body comprises a region of increased deformability relative to other parts of the main body such that when a user's head is resting on the head supporting portion, a recess, preferably a concave recess, defines a head receiving portion in an upper surface of the head supporting portion, wherein the head supporting portion comprises jaw support portions provided on opposing sides of the head receiving portion.

Preferably the main body of the pillow or sleep positioner has a generally rectangular footprint.

In an embodiment of the present invention the pillow or sleep positioner further comprises a shoulder elevating or supporting portion connected to or integral with the main body. Preferably the shoulder elevating or supporting portion is integrally formed with the main body.

Preferably the shoulder elevating or supporting portion has a substantially constant thickness. Preferably the shoulder elevating or supporting portion has a generally rectangular footprint.

In one embodiment the shoulder elevating or supporting portion comprises a tapered distal edge, wherein the tapered distal edge reduces in thickness away from the main body.

In one embodiment the main body has increased thickness relative to the shoulder supporting portion.

Preferably the shoulder elevating or supporting portion has a thickness of between around 35 mm and 65 mm. For example, the shoulder elevating or supporting portion may have a thickness of between around 35 mm and 40 mm, 40 mm and 45 mm, 45 mm and 50 mm, 50 mm and 55 mm, 55 mm and 60 mm, or 60 mm and 65 mm. For example, the shoulder elevating or supporting portion has a thickness of between around 40 mm and 60 mm. More preferably the shoulder elevating or supporting portion has a thickness of around 45 mm to 55 mm, for example around 50 mm.

Preferably the shoulder elevating or supporting portion has a density of approximately 40 kg/m³ to 100 kg/m³. For example, the density of the shoulder elevating or supporting portion may be approximately 40 kg/m³, 45 kg/m³, 50 kg/m³, 55 kg/m³, 60 kg/m³, 65 kg/m³, 70 kg/m³, 75 kg/m³, 80 kg/m³, 85 kg/m³, 90 kg/m³, 95 kg/m³, or 100 kg/m³.

In one embodiment the shoulder elevating or supporting portion may have a density that is substantially greater than that of the main body. Preferably the shoulder elevating or supporting portion may have a density that is 5% to 10% greater than that of the main body.

In one embodiment the head supporting portion comprises a recess, preferably a concave recess, provided in the lower surface of the main body and/or a cavity or void portion provided inside the main body and/or adjacent a lower surface of the main body. As a result, the part of the main body above the cavity/void portion or recess forms said region of increased deformability.

In another embodiment a recess, preferably a concave recess, is provided in the upper surface of the main body, defining a head receiving portion on an upper surface of the main body. As a result, the part of the main body below the recess forms said region of increased deformability. While such an arrangement is possible, according to preferred embodiments of the present invention the region of increased deformability is provided by means other than a concave recess provided in the upper surface of the main body.

In another embodiment a cavity or void portion is provided inside the main body or adjacent an upper surface of the main body. As a result, the part of the main body above the cavity/void portion forms said region of increased deformability.

Additionally or alternatively a portion of the main body may be formed from or provided with a material that is more easily deformable than other parts of the main body so as to define said region of increased deformability. Such other material may, for example, be provided in the previously disclosed recess or cavity/void portion and/or part of the main body may be formed from such a more easily deformable material or provided otherwise by way of insert or attachment. As will be appreciated, the pillow or sleep positioner may be integrally formed from one or more materials with the structural properties thereof varied as required.

In one embodiment the head supporting portion comprises a plurality of recesses, preferably concave recesses, provided in the lower surface of the main body and/or cavity or void portions inside the main body and/or adjacent a lower surface of the main body. Alternatively a plurality of recesses, preferably concave recesses, may be provided in the upper surface of the main body and/or a plurality of cavity or void portions may be provided inside the main body and/or adjacent an upper surface of the main body. Preferably the plurality of recesses and/or cavity or void portions are arrayed laterally across the main body.

In one embodiment, the pillow or sleep positioner comprising a plurality of cavity or void portions is a pillow or sleep positioner of an approximately standard pillow width (i.e. approximately equal to or greater than shoulder width), allowing the user to select from multiple positions on the pillow or sleep positioner. In another embodiment, the pillow or sleep positioner comprising a plurality of cavity or void portions is a pillow or sleep positioner of greater than standard width, allowing multiple users to use the pillow or sleep positioner simultaneously. For example, the pillow or sleep positioner may be approximately the full width of a standard double, queen, king, super king or California king size bed. Alternatively, the pillow or sleep positioner may have a reduced length so as to control/direct positioning of the head in the region of increased deformability. However, this must be balanced with an adequate width required for stability.

In one embodiment the head supporting portion is configured to create the jaw support portions on the head supporting portion substantially adjacent the lateral aspects of the head receiving portion on opposing sides of the head receiving portion in use. To this end the region of increased deformability is configured to reduce the ease of deformability in the jaw support portions and/or the main body is configured to have increased resistance to deformation in or about the jaw support portions. For example, the main body may be thicker in these areas (at least relative to the region of increased deformability) and/or a less deformable material may be provided, either by provision of an insert or by varying the properties of the main body in different regions.

In one embodiment the recess and/or void portion and/or cavity is bulb-shaped, comprising a bulbous head portion and a narrower neck portion, wherein the narrower neck portion is proximate to the neck supporting portion. As will be appreciated, where varying material properties are used to define the region of increased deformability, the more easily deformable material may be provided in a region defining a space similar to or the same as the recess/void portion/cavity. Preferably the bulbous head portion is adapted to receive the user's head in use. Preferably the bulbous head portion has a substantially hemispherical three-dimensional shape, but is laterally elongated. Preferably the bulbous head portion is approximately 1.05 times to 1.25 times longer along its elongated axis than along its other axis. For example, the bulbous head portion may be approximately 1.05, 1.10, 1.15 or 1.25 times longer along its elongated axis than along its other axis. More preferably the bulbous head portion is approximately 1.10 times longer along its elongated axis than along its other axis, the elongated axis being generally aligned perpendicular to the user's central axis in use.

In one particular embodiment the bulbous head portion is approximately 225 mm to 255 mm along its elongated axis and approximately 205 mm to 235 mm along its other axis. More preferably the bulbous head portion is approximately 230 mm to 250 mm along its elongated axis and approximately 210 mm to 230 mm along its other axis, or the bulbous head portion is approximately 235 mm to 245 mm along its elongated axis and approximately 215 mm to 225 mm along its other axis, for example 240 mm along its elongated axis and 220 mm along its other axis.

In other embodiments the recess and/or void portion and/or cavity may be substantially circular or ovoid. Preferably the recess and/or void portion and/or cavity is adapted to receive the user's head in use.

Preferably the recess and/or cavity/void portion may be substantially between 25 mm and 55 mm deep at its maximum when the pillow or sleep positioner is at rest. For example, the void space and/or recess may be substantially 25 mm, 30 mm, 35 mm, 40 mm, 45 mm, 50 mm, 55 mm deep at its maximum when the pillow or sleep positioner is at rest. Preferably the recess and/or cavity/void space may be substantially 25 mm to 45 mm deep at its maximum when the pillow or sleep positioner is at rest. More preferably the recess and/or cavity/void space may be substantially 30 mm to 40 mm deep at its maximum when the pillow or sleep positioner is at rest, for example 35 mm deep at its maximum when the pillow or sleep positioner is at rest.

In one particular embodiment the main body may be manufactured from latex and the recess and/or void space may be substantially 25 mm to 45 mm deep at its maximum when the pillow or sleep positioner is at rest. Preferably the recess and/or void space may be substantially 30 mm to 40 mm deep at its maximum when the pillow or sleep positioner is at rest, for example 35 mm deep at its maximum when the pillow is at rest.

In one embodiment the cavity/void portion and/or recess may contain a light filler material.

Preferably the head supporting portion is adapted to support the user's head in a substantially horizontal position when the user is lying on their back.

Preferably the head supporting portion is adapted to support the user's head in a neutral position when the user is lying on their side.

In one embodiment the head supporting portion comprises a first end region and a second end region, wherein the first end region is proximate to the user's shoulders in use and the second end region is distal to the user's shoulders in use.

Preferably the head supporting portion has a slight incline from the first end region to the second end region, away from the neck supporting portion, such that the second end portion of the head supporting region is higher relative to the first end portion. Preferably the incline may be between approximately 1° and 10°. For example, the incline may be approximately 1° to 5° or 5° to 10°. For example, the incline may be between approximately 2° to 9°, 3° to 8°, or 4° to 6°.

Preferably the neck supporting portion has a height of between 85 mm and 125 mm above an upper surface of the shoulder elevating or supporting portion if present or above the surface the user lies on if the shoulder elevating or supporting portion is not present. For example, the neck supporting portion may have a height of between 85 mm and 90 mm, between 90 mm and 95 mm, between 95 mm and 100 mm, between 100 mm and 105 mm, between 105 mm and 110 mm, between 110 mm and 115 mm, between 115 mm and 120 mm, or between 120 mm and 125 mm above an upper surface of the shoulder elevating or supporting portion if present or above the surface the user lies on if the shoulder elevating or supporting portion is not present. For example, the neck supporting portion may have a height of between 90 mm and 120 mm or between 95 mm and 115 mm. More preferably the neck supporting portion has a height of between 100 mm and 120 mm above an upper surface of the shoulder elevating or supporting portion if present or above the surface the user lies on if the shoulder elevating or supporting portion is not present, for example 110 mm above an upper surface of the shoulder elevating or supporting portion if present or above the surface the user lies on if the shoulder elevating or supporting portion is not present.

In one embodiment of the present invention the neck supporting portion comprises a ridge or lip (hereafter referred to as a ridge). Preferably the ridge is situated substantially on the upper surface of the neck supporting portion. More preferably the ridge is situated substantially on the upper surface of the neck supporting portion and substantially adjacent the top edge of the shoulder-facing surface of the neck supporting portion.

Preferably the ridge is adapted to support the user's jaw when the user is lying on their side such that the user's head is supported in a neutral position, i.e. the user is supported in a position where there is substantially reduced lateral flexion of the cervical spine relative to a neutral head carriage position when the user is lying on their side relative to lying on their side without the pillow or sleep positioner.

In one embodiment the ridge may be approximately 65 mm to 95 mm wide from the top edge of the shoulder-facing surface of the neck supporting portion toward the opposite edge of the main body. For example, the ridge may be approximately 65 mm to 70 mm wide, 70 mm to 75 mm wide, 75 mm to 80 mm wide, 80 mm to 85 mm, 85 mm to 90 mm or 90 mm to 95 mm wide. For example, the ridge may be approximately 70 mm to 90 mm wide, 75 mm to 85 mm wide, or 70 mm to 80 mm wide, for example 80 mm wide.

In one embodiment the ridge may have a height of approximately 5 mm to 15 mm.

In one embodiment of the present invention, the top edge and the shoulder-facing surface of the neck supporting portion define a contour having a medial concave portion and lateral convex portions on opposing sides.

Preferably the midpoint of the medial concave portion of the contour is substantially aligned with the midpoint of the recess and/or cavity/void portion.

Preferably the medial concave portion of the contour is adapted to receive the user's shoulder when the user is lying on their side.

Preferably the lateral convex portions of the contour are adapted to receive the user's jaw on the top shoulder-facing edge when the user is lying on their side such that the user's head is supported in a neutral position.

Preferably, the medial concave portion of the contour has a maximum depth of approximately 20 mm to 50 mm. For example, the medial concave portion of the contour may have a maximum depth of approximately 20 mm to 25 mm, approximately 25 mm to 30 mm, approximately 30 mm to 35 mm, approximately 35 mm to 40 mm, approximately 40 mm to 45 mm or approximately 45 mm to 50 mm. For example, the medial concave portion of the contour may have a maximum depth of approximately 25 mm to 50 mm or approximately 30 mm to 45 mm. Preferably the medial concave portion of the contour may have a maximum depth of approximately 30 mm to 40 mm, for example 35 mm.

In a particularly preferred embodiment of the present invention the ridge and contour are adapted to act in concert to assist in causing the user's head to be held substantially in line with the natural or neutral position, and in particular adapted to assist in causing the user's jaw to be slightly elevated when the user is lying on their side relative to its position if the configuration is absent, thus causing the user's jaw to be held in a neutral position. In such an embodiment the ridge substantially follows the shape of the contour.

In one embodiment of the present invention the head supporting portion comprises a plurality of recesses and/or void portions and the shoulder-facing surface of the neck supporting portion defines a plurality of contours having medial concave portions and lateral convex portions on opposing sides, in the manner of a sinusoidal wave form. Preferably the midpoint of each of the concave portions of the plurality of contours is substantially aligned with the midpoint of each of the plurality of recesses and/or void portions.

In an embodiment of the present invention the main body comprises a suitably resiliently compressible material.

Preferably the main body comprises latex. More preferably, the main body comprises latex comprising pinholes capable of varying the density of the latex over the different portions of the main body. For example, the density of the latex may be varied by the number, depth, spacing and/or size of pinholes therein.

Preferably the main body has a density of approximately 40 kg/m³ to 100 kg/m³. For example, the density of the main body may be approximately 40 kg/m³, 45 kg/m³, 50 kg/m³, 55 kg/m³, 60 kg/m³, 65 kg/m³, 70 kg/m³, 75 kg/m³, 80 kg/m³, 85 kg/m³, 90 kg/m³, 95 kg/m³ or 100 kg/m³.

In one embodiment, if the shoulder supporting or elevating portion is present, the main body may have a density substantially lower than that of the shoulder supporting or elevating portion.

In one embodiment of the present invention the main body may comprise a layer or stratum substantially adjacent the lower surface which has a density substantially greater than that of the remainder of the main body. In one embodiment this layer or stratum of the main body may be integral with the shoulder supporting or elevating portion, although the invention is not limited thereto.

Preferably the main body comprises a plurality of recesses and/or cavity/void portions which define a plurality of head receiving portions in use.

In one embodiment of the present invention the main body of the pillow or sleep positioner may be wider and/or longer than a normal pillow in order to accommodate a user of high body weight, such as an overweight, obese or morbidly obese user. In such an embodiment, if the shoulder supporting or elevating portion is present, the shoulder supporting or elevating portion may be increased in size, preferably substantially proportionately. Alternatively, the main body of the pillow or sleep positioner may be substantially the size of a normal pillow and the shoulder elevating or supporting portion increased in size to accommodate the user of high body weight.

In one embodiment of the present invention the upper surface of the main body and/or the upper surface of the shoulder supporting or elevating portion of the pillow or sleep positioner may comprise or be provided with a comfort layer providing a desirable “feel” to the user. For example, such a layer may comprise one or more material that is softer or more deformable than the main body and/or shoulder supporting or elevating portion or such a layer may comprise one or more breathable material, or a combination thereof.

In one embodiment the pillow or sleep positioner may comprise one or more sensor means for generating signals in response to a user's body motion and/or for generating biometric signals indicative of biological activity in the user's body and/or for generating signals in response to environmental factors. The one or more sensor means may be provided within the pillow or sleep positioner or may be located on the surface of the pillow or sleep positioner. The one or more sensor means may be integral with a suitable fabric cover for the pillow or sleep positioner, i.e. a smart fabric, or otherwise provided on or in the pillow or sleep positioner or integrated therewith.

The sensor signals may be processed to monitor factors having a positive or negative impact on the user's sleep. Data from the sensor signals may be communicated to other sensors, processors, data storage, I/O systems, power systems, communication interfaces etc. Data may be transferred wirelessly such, by way of non-limiting example, via WiFi or Bluetooth, or via a wired communication link or interface. Data from the sensor signals may be presented to the user via a client device such as a PC, laptop, smart phone, smart watch, or tablet etc. that includes an application. Data may be displayed in the form of a graphical user interface.

Non-limiting examples of sensors that may be suitable for use in the present invention include sensors for measuring heart rate, respiratory rate, heart rate variability, body temperature, bioimpedence, blood oxygen, skin conductance, electromyography, electroencephalography, motion (accelerometers, gyroscopes etc.), ambient temperature and/or humidity, sound, light etc.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

According to a still further aspect of the present invention, a pillow or sleep positioner is substantially as herein described is provided, with reference to the accompanying drawings. Further aspects of the invention, which should be considered in all its novel aspects, will become apparent from the following description given by way of example of possible embodiments of the invention.

### BRIEF DESCRIPTION OF THE DRAWINGS

FIG. 1 is a top perspective view of a pillow according to one embodiment of the invention, with a recess shown in hidden detail and the fabric cover removed for clarity.

FIG. 2 is a side view of the pillow of FIG. 1, with the recess shown in hidden detail and the fabric cover removed for clarity.

FIG. 3 is a top view of the pillow of FIG. 1, with the recess shown in hidden detail and the fabric cover removed for clarity.

FIG. 4 is an isometric view of the pillow of FIG. 1 from below, with the fabric cover removed for clarity.

FIG. 5 is a diagrammatic side view of the pillow of FIG. 1 in use, with a user's head shown diagrammatically in outline and the fabric cover removed for clarity.

FIG. 6 is a top perspective view of a pillow according to another embodiment of the invention, with the fabric cover removed for clarity and with pinholes shown in representative form.

FIG. 7 is a top perspective view of the pillow of FIG. 6, with the recess shown in hidden and the detail fabric cover removed for clarity. For clarity, pinholes are not shown.

FIG. 8 is a side view of the pillow of FIG. 6, with the fabric cover removed for clarity.

FIG. 9 is a top view of the pillow of FIG. 6, with the fabric cover removed for clarity and with pinholes shown in representative form.

FIG. 10 is a top view of the pillow of FIG. 6, with the recess shown in hidden detail and fabric cover removed for clarity. For clarity, pinholes are not shown.

FIG. 11 is a section view of the pillow of FIG. 9 through A-A, with the recess shown in hidden detail, with the fabric cover removed for clarity and with pinholes shown in representative form.

FIG. 12 is an isometric view of the pillow of FIG. 6 from below, with the fabric cover removed for clarity and with pinholes shown in representative form.

### DEFINITIONS

The phrase “at rest” is used herein to indicate the state of the pillow or sleep positioner when not deformed by the weight of a user.

The phrase “central axis” is used herein to indicate the axis running through the midpoint of a user's head and body in a superior-inferior direction through the midpoint of a user's head, i.e. the vertical axis when the user is in the anatomical position.

The phrase “recess” is used herein to indicate a portion on the surface of the pillow or sleep positioner which is recessed relative to the otherwise flush surface of the pillow.

The phrases “neutral head position”, “natural sleeping position”, and the like are used herein to indicate the user being in a position where their head is substantially horizontal, having substantially minimal forward flexion or extension of the cervical spine relative to a neutral head carriage position when the user is lying on their back, and to indicate the user being in a position where there is substantially reduced lateral flexion of the cervical spine relative to a neutral head carriage position when the user is lying on their side relative to lying on their side without the pillow or sleep positioner.

The phrase “shoulder-facing surface” is used herein to indicate the surface of the pillow or sleep positioner that abuts the user's shoulders in use.

The phrases “cavity” and “void portion” are used herein to indicate a substantially hollow space, cavity, hole or other suitable void space located within the main body, i.e. “cavity” and “void portion” are distinguished from “recess” in that the hollow space is internal to the main body.

### BRIEF DESCRIPTION OF PREFERRED EMBODIMENTS OF THE INVENTION

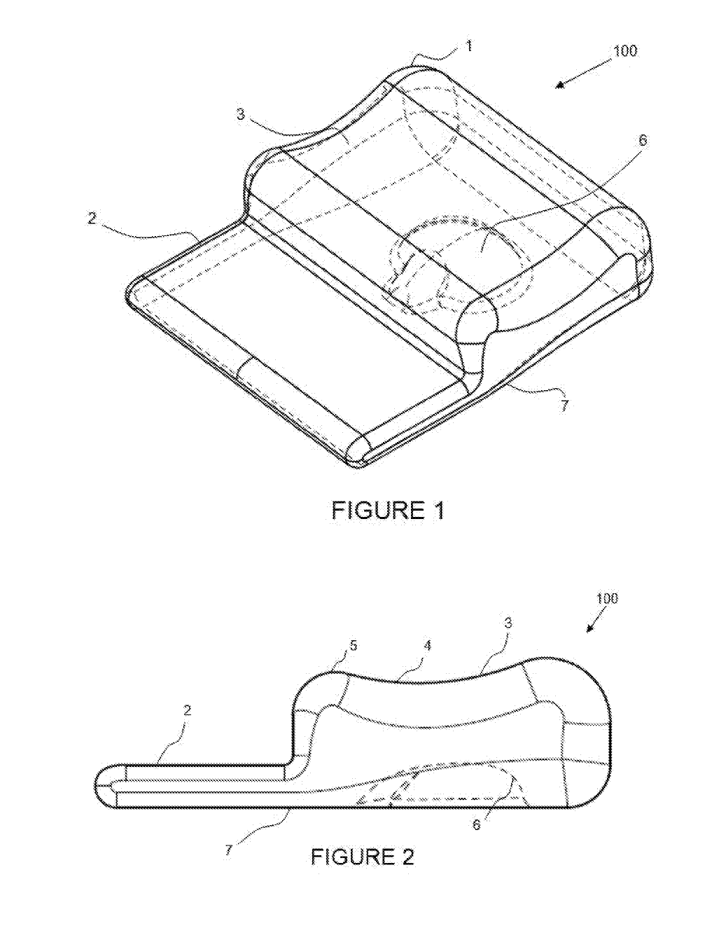

Referring to FIGS. 1 to 5, a pillow according to one embodiment of the present invention is generally referenced by arrow 100.

The pillow 100 comprises a main body 1 with a shoulder elevating or supporting portion 2 connected to or integrally formed with the main body 1. The main body 1 and shoulder elevating or supporting portion 2 are preferably manufactured from a suitably resiliently compressible material such as, for example but not limited to, latex, and are covered by a suitably breathable fabric (not shown). Further non-limiting examples of suitably resiliently compressible material include foam, memory foam, viscoelastic polyurethane foam, low-resilience polyurethane foam, gels, gel foams, natural fibre (such as cotton, wool, feathers etc.) and/or synthetic fibre (such as polyester, nylon, rayon, acetate etc.).

Preferably the main body 1 has a density of approximately 40 kg/m³ to 100 kg/m³. For example, the density of the main body 1 may be approximately 40 kg/m³, 45 kg/m³, 50 kg/m³, 55 kg/m³, 60 kg/m³, 65 kg/m³, 70 kg/m³, 75 kg/m³, 80 kg/m³, 85 kg/m³, 90 kg/m³, 95 kg/m³ or 100 kg/m³.

Different densities may be achieved through the use of different materials, non-limiting examples of which include those listed above. Other suitable materials will be readily apparent to a person skilled in the art.

In the embodiment shown in FIGS. 1 to 5 the optional shoulder elevating or supporting portion 2 is present. The shoulder elevating or supporting portion 2 is preferably around 35 mm to 65 mm thick, more preferably around 45 mm to 55 mm thick when at rest, for example 50 mm thick when at rest, and preferably elevates the user's shoulder around 20 mm to 35 mm above the surface of the bed or any other sleeping surface in use, depending on the weight of the user and the density of the material used to construct the shoulder elevating or supporting portion. The shoulder elevating or supporting portion 2 is preferably of substantially uniform thickness when at rest. This configuration assists in maintaining the user in a natural sleeping position, with their airway open.

Preferably the shoulder elevating or supporting portion 2 has a density of approximately 40 kg/m³ to 100 kg/m³. For example, the density of the shoulder elevating or supporting portion 2 may be approximately 40 kg/m³, 45 kg/m³, 50 kg/m³, 55 kg/m³, 60 kg/m³, 65 kg/m³, 70 kg/m³, 75 kg/m³, 80 kg/m³, 85 kg/m³, 90 kg/m³, 95 kg/m³ and 100 kg/m³.

In certain embodiments the shoulder elevating or supporting portion 2 may have a greater density that that of the main body 1. Preferably the shoulder elevating or supporting portion may have a density that is 5% to 10% greater than that of the main body. Different densities may be achieved through the use of different materials, non-limiting examples of which include those listed above. Other suitable materials and methods for varying density of the different portions of the pillow, where desirable, will be readily apparent to a person skilled in the art.

An upper surface 3 of the main body 1 comprises a head supporting portion 4 and a neck supporting portion 5, as is apparent from FIG. 2 for example. The head supporting portion 4 comprises a first end region and a second end region, wherein the first end region is proximate to the user's shoulders in use and the second end region is distal to the user's shoulders in use.

The head supporting portion 4 preferably has a slight incline from the first end region to the second end region, away from the neck supporting portion 5, such that the second end portion of the head supporting region is higher relative to the first end region. Preferably, the incline may be between approximately 1° and 10°. For example, but not limited to, the incline may be approximately 1° to 5° or 5° to 10°. For example, the incline may be between approximately 2° to 9°, 3° to 8°, or 4° to 6°.

The neck supporting portion 5 is preferably around 85 mm to 125 mm above the shoulder elevating or supporting portion 2 (if present) when at rest. For example, the neck supporting portion 5 may be around 85 mm to 90 mm, 90 mm to 95 mm, 95 mm to 100 mm, 100 mm to 105 mm, 105 mm to 110 mm, 110 mm to 115 mm, 115 mm to 120 mm, or 120 mm to 125 mm above the shoulder elevating or supporting portion 2 (if present) when at rest. For example, the neck supporting portion 5 may be around 90 mm to 120 mm or 95 mm to 115 mm above the shoulder elevating or supporting portion 2 (if present) when at rest, for example around 110 mm above the shoulder elevating or supporting portion 2 (if present) when at rest. In embodiments where the shoulder elevating or supporting portion 2 is not present, the neck supporting portion 5 is preferably around 85 mm to 125 mm above the bed or other sleeping surface when at rest. For example, the neck supporting portion 5 may be around 85 mm to 90 mm, 90 mm to 95 mm, 95 mm to 100 mm, 100 mm to 105 mm, 105 mm to 110 mm, 110 mm to 115 mm, 115 mm to 120 mm, or 120 mm to 125 mm above the bed or other sleeping surface when at rest. For example, the neck supporting portion 5 may be around 90 mm to 120 mm or 95 mm to 115 mm above the bed or other sleeping surface when at rest, for example around 110 mm above the bed or other sleeping surface when at rest.

The main body 1 comprises a region of increased deformability relative to other parts of the main body 1 such that when a user's head H is resting on the head supporting portion 4 a recess, preferably a concave recess, defines a head receiving portion in an upper surface of the head supporting portion 4. In the embodiment shown a concave recess 6 is provided in a lower surface 7 of the main body. As will be appreciated, where varying material properties are used to define the region of increased deformability, the more easily deformable material may be provided in a region defining a space similar to or the same as the recess/void portion/cavity.

In the embodiment shown in FIGS. 1 to 5 the recess 6 is substantially concave and bulb-shaped, comprising a wider bulbous head portion 10 and a narrower neck portion 11, wherein the narrower neck portion 11 is proximate to the neck supporting portion 5. Preferably the bulbous head portion 10 is adapted to receive the user's head in use.

In some embodiments the bulbous head portion 10 is substantially hemispherical in shape, but is laterally elongated. Preferably the bulbous head portion is approximately 1.05 times to 1.25 times longer along its elongated axis than along its other axis. For example, the bulbous head portion may be approximately 1.05, 1.10, 1.15 or 1.25 times longer along its elongated axis than along its other axis. More preferably the bulbous head portion is approximately 1.10 times longer along its elongated axis than along its other axis, the elongated axis being generally aligned perpendicular to the user's central axis in use.

In some embodiments the bulbous head portion is approximately 225 mm to 255 mm along its elongated axis and approximately 205 mm to 235 mm along its other axis. For example, the bulbous head portion is approximately 230 mm to 250 mm along its elongated axis and approximately 210 mm to 230 mm along its other axis, or the bulbous head portion is approximately 235 mm to 245 mm along its elongated axis and approximately 215 mm to 225 mm along its other axis, for example 240 mm along its elongated axis and 220 mm along its other axis.

In preferred embodiments the recess 6 (or cavity/void portion) is substantially 25 mm to 55 mm deep at its maximum when the pillow is at rest. For example, the recess 6 (or cavity/void portion) may be substantially 25 mm to 30 mm deep at its maximum, 30 mm to 35 mm deep at its maximum, substantially 35 mm to 40 mm deep at its maximum, substantially 40 mm to 45 mm deep at its maximum, substantially 45 mm to 50 mm deep at its maximum or substantially 50 mm to 55 mm deep at its maximum when the pillow is at rest. Preferably the recess and/or cavity/void portion may be substantially 25 mm to 45 mm deep at its maximum when the pillow is at rest. More preferably the recess and/or cavity/void space may be substantially 30 mm to 40 mm deep at its maximum when the pillow is at rest, for example 35 mm deep at its maximum when the pillow is at rest.

In the embodiment shown in FIGS. 1 to 5 the recess 6 (or cavity/void portion) is shaped such that when a user rests their head H on the head supporting portion 4, a head receiving portion 8 is defined in an upper surface of the head supporting portion 4.

In embodiments where the recess 6 (or cavity/void portion) is provided in the lower surface 7 (or adjacent the lower surface 7) of the main body 1, the head receiving portion 8 is formed by the weight of the user's head H compressing the recess 6 or cavity/void portion. As a result, the part of the main body 1 above the recess or cavity/void portion forms a region of increased deformability relative to other parts of the main body. The recess 6 or cavity/void portion may be completely collapsed by the weight of the user's head, but more preferably is only partially or mostly compressed. Embodiments in which the recess 6 or cavity/void portion are not completely compressed may be more comfortable for the user, and in particular, may reduce the pressure felt on the user's ear when they are lying on their side. More particularly, embodiments in which the narrower neck portion 11 of the concave recess 6 is not completely compressed may reduce the pressure felt on the user's ear when they are lying on their side.

In a further embodiment of the present invention, a light filler material may be included in the recess 6 or cavity/void portion. Inclusion of a light filler material may assist with avoiding complete collapse of the recess 6 or cavity/void portion.

In embodiments where a recess 6 is provided in the upper surface 3 of the main body 1, the recess 6 may define a head receiving portion 8 in an upper surface of the head supporting portion 4. As a result, the part of the main body below the recess forms a region of increased deformability relative to other parts of the main body.

In embodiments where a cavity/void portion is provided adjacent the upper surface 3 of the main body 1, the head receiving portion 8 is formed by the weight of the user's head H compressing the cavity/void portion. As a result, the part of the main body 1 above the cavity/void portion forms a region of increased deformability relative to other parts of the main body. The recess 6 or cavity/void may be completely collapsed by the weight of the user's head, but more preferably is only partially or mostly compressed.

Embodiments of the present invention in which the recess 6 (or cavity/void portion) is provided in a lower surface 7 (or adjacent a lower surface) of the main body 1 may be more comfortable than pillows of the prior art in which a recess is formed in the upper surface 3 of the pillow, as the transition between the head receiving portion 8 and the remainder of the head supporting portion 4 may be softened. This means that the user is not forced to place their head exactly in a predetermined head receiving portion.

Embodiments of the present invention in which the recess 6 (or cavity/void portion) is provided in an upper surface 3 (or adjacent an upper surface 3) of the main body 1 may be suitable for applications where the retention of the user's head in a substantially fixed position is desirable as the recess or cavity/void portion may tend to hold the user's head more fixedly when it is provided in (or adjacent) an upper surface 3 of the main body 1. In embodiments where a recess 6 is provided in the upper surface of the main body 1 the recess 6 may define a head receiving portion on an upper surface of the main body. For example, such embodiments of the present invention may be suitable for use in surgical or clinical settings or where the user suffers from a sleep disorder characterised by pauses in breathing or instances of shallow breathing during sleep such as sleep apnoea.

In other embodiments a portion of the main body may be formed from or provided with a material that is more easily deformable than other parts of the main body so as to define a region of increased deformability. Such other material may, for example, be provided in the previously disclosed recess or cavity/void portion and/or part of the main body may be formed from such a more easily deformable material or provide otherwise by way of insert or attachment. As will be appreciated, the pillow may be integrally formed from one or more materials with the structural properties thereof varied as required.

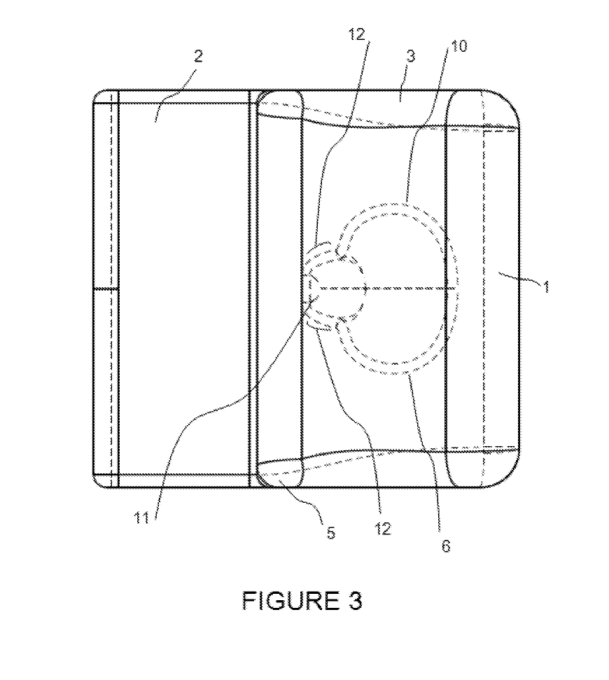

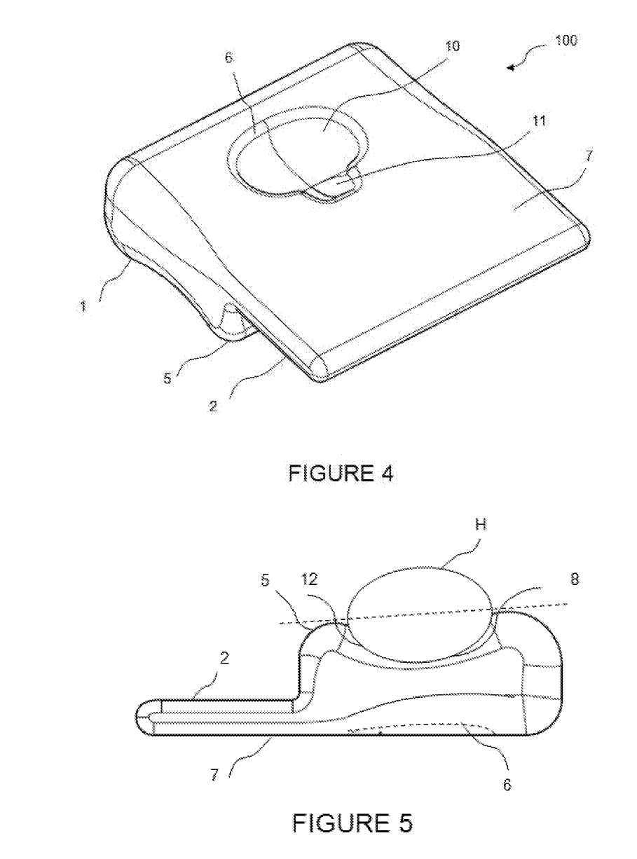

As is best seen in FIGS. 3 and 4, the recess 6 (or cavity/void portion) may comprise a wider portion 10 and a narrower neck portion 11. The narrower neck portion 11 is closer to the shoulder elevating or supporting portion 2 (if present as shown in FIGS. 3 and 4) than the wider portion 10. The wider portion 10 is substantially hemispherical in shape, but is elongated in the width direction of the pillow 100. The recess 6 (or cavity/void portion) is substantially bulb-shaped, having a wider bulbous head portion 10 and a narrower neck portion 11. The narrower neck portion 11 preferably slopes towards the lower surface of the pillow from the intersection of the narrower neck portion 11 with the wider portion 10.

The head supporting portion 6 is shaped to create a jaw support portion 12 on either side of the head receiving portion 8 in use. The jaw support portions 12 ensure that the user's jaw is held slightly higher than it would be if these portions were not present. In the pillows of the prior art the user's chin may tuck downward toward the chest when the user is lying on their side, thereby occluding their airway.

In other embodiments the head supporting portion is configured to create the jaw support portions on the head supporting portion substantially adjacent the lateral aspects of the head receiving portion on opposing sides of head receiving portion in use. To this end the region of increased deformability is configured to reduce the ease of deformability in the jaw support portions and/or the main body is configured to have increased resistance to deformation in or about the jaw support portions. For example, the main body may be thicker in these areas (at least relative to the region of increased deformability) and/or a less deformable material may be provided, either by provision of an insert or by varying the properties of the main body in different regions.

In use, the user's head H and neck are held in a substantially neutral position. As seen in FIG. 5, the jaw support portions 12 combine with the head receiving portion 8 to cause the user's head to be held substantially in line with the natural or neutral position of the user's head. Therefore, when the user is lying on their side a central axis through the user's head, from neck to crown, is almost horizontal, but may slope upward very slightly. This opens the user's airways and reduces snoring when the user is lying on their side.

When on their back, the central axis is substantially horizontal, and an axis from the back of the user's head to the front of the user's head is substantially vertical. This straight and natural positioning of the user's head keeps airways open and reduces snoring.

In another embodiment (not shown), the pillow 100 may be wider than usual, and two (or more) concave recesses (or void spaces) may be provided side-by-side, so that the user may choose from a variety of positions, or, in the case of full width pillows, so that multiple users may use the pillow at the same time.

In other embodiments (not shown) the shoulder elevating or supporting portion 2 may be omitted, and the height of the upper surface of the pillow reduced slightly to compensate.

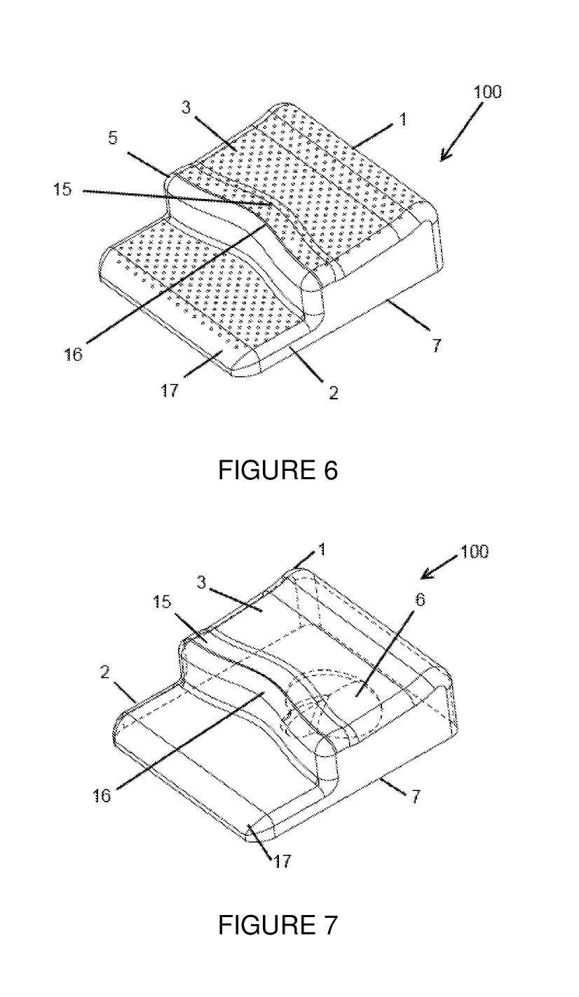

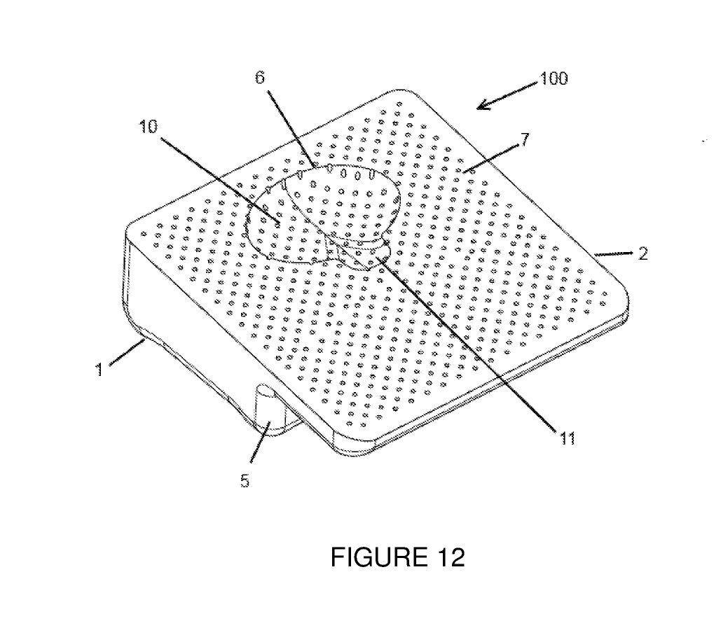

Referring to FIGS. 6 to 12, a pillow according to one embodiment of the present invention is generally referenced by arrow 100.

The pillow 100 comprises a main body 1 with a shoulder elevating or supporting portion 2 connected to or integrally formed with the main body 1. The main body 1 and shoulder elevating or supporting portion 2 are preferably manufactured from a suitably resiliently compressible material such as, for example but not limited to, latex, and are covered by a suitably breathable fabric (not shown). Further non-limiting examples of suitably resiliently compressible material include foam, memory foam, viscoelastic polyurethane foam, low-resilience polyurethane foam, gels, gel foams, natural fibre (such as cotton, wool, feathers etc.) and/or synthetic fibre (such as polyester, nylon, rayon, acetate etc.).

The main body 1 and shoulder elevating or supporting portion 2 may be manufactured from latex comprising pinholes capable of varying the density of the latex. In FIGS. 6, 8, 9, 11 and 12 pinholes are shown in representative form.

The pinholes may vary in number, diameter, spacing and/or depth, effecting concomitant variation in the overall density of the latex. For example, if greater density is desired then fewer, smaller diameter, more spaced apart, and/or shallower pinholes may be introduced to the latex. For example, if lower density is desired the more, larger diameter, more closely spaced and/or deeper pinholes may be introduced to the latex. In one embodiment, the pinholes may be configured so as to create a layer or stratum within the main body 1 and/or shoulder elevating or supporting portion 2 that is not penetrated by the pinholes. In such an embodiment the layer or stratum has a substantially greater density than that of the surrounding areas that are penetrated by the pinholes. By way of non-limiting example, the layer or stratum may have a density that is approximately 5% to 10% greater than that of the surrounding areas that are penetrated by the pinholes.

Pinholes as described herein can also be provided in other embodiments of the present invention.

In one embodiment the main body may comprise a layer or stratum substantially adjacent the lower surface which has a density substantially greater than that of the remainder of the main body. In one embodiment this layer or stratum of the main body may be integral with the shoulder supporting or elevating portion, although the invention is not limited thereto. This stratum or layer may be formed of or provided with a different material that is less easily deformable than other parts of the main body. Additionally or alternatively, the pillow may be integrally formed from one or more materials with the structural properties thereof varied as required.

Preferably the main body 1 may have a density of approximately 40 kg/m³ to 100 kg/m³. For example, the density of the main body 1 may be approximately 40 kg/m³, 45 kg/m³, 50 kg/m³, 55 kg/m³, 60 kg/m³, 65 kg/m³, 70 kg/m³, 75 kg/m³, 80 kg/m³, 85 kg/m³, 90 kg/m³, 95 kg/m³ or 100 kg/m³.

Different densities may be achieved through the use of different materials, non-limiting examples of which include those listed above. Other suitable materials will be readily apparent to a person skilled in the art.

In the embodiment shown in FIGS. 6 to 12 the optional shoulder elevating or supporting portion 2 is present. The shoulder elevating or supporting portion 2 is preferably around 35 mm to 65 mm thick, more preferably around 25 mm to 55 mm thick when at rest, for example 50 mm, thick when at rest, and preferably elevates the user's shoulder around 20 mm to 35 mm above the surface of the bed or any other sleeping surface in use, depending on the weight of the user and the density of the material used to construct the shoulder elevating or supporting portion. The shoulder elevating or supporting portion 2 is preferably of substantially uniform thickness when at rest. This configuration assists in maintaining the user in a natural sleeping position, with their airway open.

Preferably the shoulder elevating or supporting portion 2 may have a density of approximately 40 kg/m³ to 100 kg/m³. For example, the density of the shoulder elevating or supporting portion 2 may be approximately 40 kg/m³, 45 kg/m³, 50 kg/m³, 55 kg/m³, 60 kg/m³, 65 kg/m³, 70 kg/m³, 75 kg/m³, 80 kg/m³, 85 kg/m³, 90 kg/m³, 95 kg/m³, or 100 kg/m³.

In certain embodiments the shoulder elevating or supporting portion 2 may have a greater density than that of the main body 1. Preferably the shoulder elevating or supporting portion may have a density that is 5% to 10% greater than that of the main body. Different densities may be achieved through the use of different materials, non-limiting examples of which include those listed above. Other suitable materials and methods for varying density of the different portions of the pillow, where desirable, will be readily apparent to a person skilled in the art.

In the embodiment shown in FIGS. 6 to 12 the shoulder elevating or supporting portion comprises a tapered distal edge 17 wherein the tapered distal edge 17 reduces in thickness away from the main body 1. The tapered distal edge 17 assists in providing the user with a comfortable interface between shoulder elevating or supporting portion 2 and the bed or other sleeping surface.

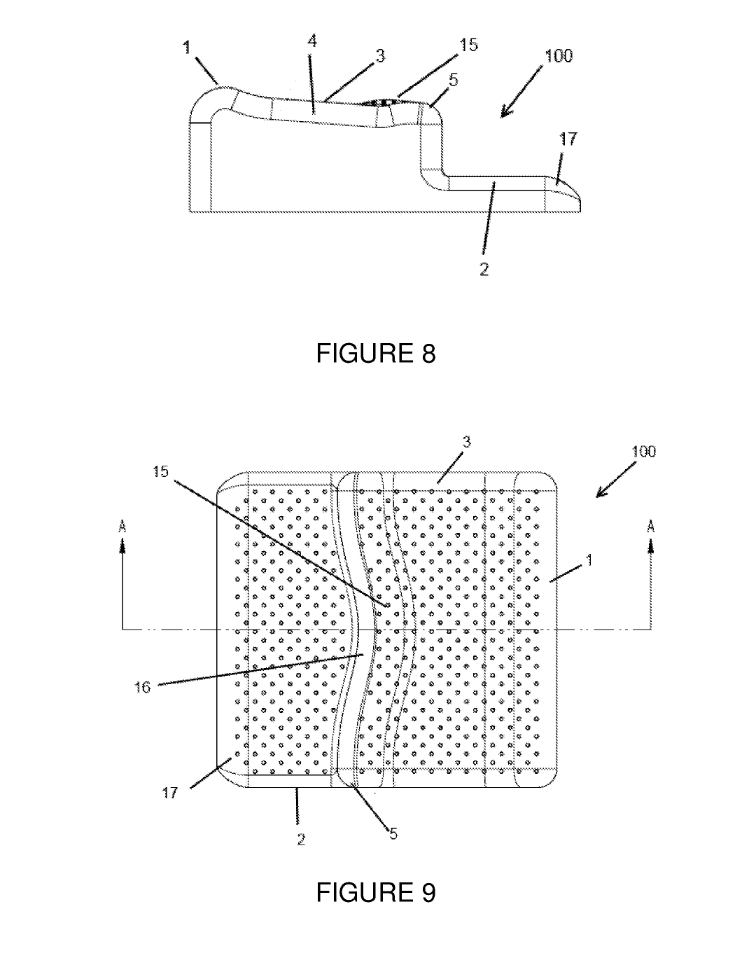

An upper surface 3 of the main body 1 comprises a head supporting portion 4 and a neck supporting portion 5, as is apparent from FIG. 8 for example. The head supporting portion 4 comprises a first end region and a second end region, wherein the first end region is proximate to the user's shoulders in use and the second end region is distal to the user's shoulders in use.

The head supporting portion 4 preferably has a slight incline from the first end region to the second end region, away from the neck supporting portion 5, such that the second end portion of the head supporting region is higher relative to the first end region. Preferably the incline may be between approximately 1° and 10°. For example, the incline may be approximately 1° to 5° or 5° to 10°. For example, the incline may be between approximately 2° to 9°, 3° to 8°, or 4° to 6°.

The neck supporting portion 5 is preferably around 85 mm to 125 mm above the shoulder elevating or supporting portion 2 (if present) when at rest. For example, the neck supporting portion 5 may be around 85 mm to 90 mm, 90 mm to 95 mm, 95 mm to 100 mm, 100 mm to 105 mm, 105 mm to 110 mm, 110 mm to 115 mm, 115 mm to 120 mm, or 120 mm to 125 mm above the shoulder elevating or supporting portion 2 (if present) when at rest. For example, the neck supporting portion 5 may be around 90 mm to 120 mm or 95 mm to 115 mm above the shoulder elevating or supporting portion 2 (if present) when at rest, for example around 110 mm above the shoulder elevating or supporting portion 2 (if present) when at rest.

In embodiments where the shoulder elevating or supporting portion 2 is not present, the neck supporting portion 5 is preferably around 85 mm to 125 mm above the bed or other sleeping surface when at rest. For example, the neck supporting portion 5 may be around 85 mm to 90 mm, 90 mm to 95 mm, 95 mm to 100 mm, 100 mm to 105 mm, 105 mm to 110 mm, 110 mm to 115 mm, 115 mm to 120 mm, or 120 mm to 125 mm above the bed or other sleeping surface when at rest. For example, the neck supporting portion 5 may be around 90 mm to 120 mm or 95 mm to 115 mm above the bed or other sleeping surface when at rest, for example around 110 mm above the bed or other sleeping surface when at rest.

The main body 1 comprises a region of increased deformability relative to other parts of the main body 1 such that when a user's head H is resting on the head supporting portion 4 a recess, preferably a concave recess, defines a head receiving portion in an upper surface of the head supporting portion 4. As will be appreciated, where varying material properties are used to define the region of increased deformability, the more easily deformable material may be provided in a region defining a space similar to or the same as the recess/void portion/cavity.

In the embodiment shown a concave recess 6 is provided in a lower surface 7 of the main body. In the embodiment shown in FIGS. 6 to 12 the recess 6 is substantially concave and bulb-shaped, comprising a wider bulbous head portion 10 and a narrower neck portion 11, wherein the narrower neck portion 11 is proximate to the neck supporting portion 5. Preferably the bulbous head portion 10 is adapted to receive the user's head in use.

In some embodiments the bulbous head portion 10 is substantially hemispherical in shape, but is laterally elongated. Preferably the bulbous head portion is approximately 1.05 times to 1.25 times longer along its elongated axis than along its other axis. For example, the bulbous head portion may be approximately 1.05, 1.10, 1.15 or 1.25 times longer along its elongated axis than along its other axis. More preferably the bulbous head portion is approximately 1.10 times longer along its elongated axis than along its other axis, the elongated axis being generally aligned perpendicular to the user's central axis in use.

In some embodiments the bulbous head portion is approximately 225 mm to 255 mm along its elongated axis and approximately 205 mm to 235 mm along its other axis. For example the bulbous head portion is approximately 230 mm to 250 mm along its elongated axis and approximately 210 mm to 230 mm along its other axis, or the bulbous head portion is approximately 235 mm to 245 mm along its elongated axis and approximately 215 mm to 225 mm along its other axis, for example 240 mm along its elongated axis and 220 mm along its other axis.

In preferred embodiments the recess 6 (or cavity/void portion) is substantially 25 mm to 55 mm deep at its maximum when the pillow is at rest. For example, the recess 6 (or cavity/void portion) may be substantially 25 mm to 30 mm deep at its maximum, substantially 30 mm to 35 mm deep at its maximum, substantially 35 mm to 40 mm deep at its maximum, substantially 40 mm to 45 mm deep at its maximum, substantially 45 mm to 50 mm deep or substantially 50 mm to 55 mm deep at its maximum when the pillow is at rest. Preferably the recess and/or cavity/void portion may be substantially 25 mm to 45 mm deep at its maximum when the pillow is at rest. More preferably the recess and/or cavity/void space may be substantially 30 mm to 40 mm deep at its maximum when the pillow is at rest, for example 25 mm deep at its maximum when the pillow is at rest.

In the embodiment shown in FIGS. 6 to 12 the recess 6 (or cavity/void portion) is shaped such that when a user rests their head H on the head supporting portion 4, a head receiving portion 8 is defined in an upper surface of the head supporting portion 4.

In embodiments where the recess 6 (or cavity/void portion) is provided in the lower surface 7 (or adjacent the lower surface 7) of the main body 1, the head receiving portion 8 is formed by the weight of the user's head H compressing the recess 6 or cavity/void portion. As a result, the part of the main body 1 above the recess or cavity/void portion forms a region of increased deformability relative to other parts of the main body. The recess 6 or cavity/void portion may be completely collapsed by the weight of the user's head, but more preferably is only partially or mostly compressed. Embodiments in which the recess 6 or cavity/void portion are not completely compressed may be more comfortable for the user, and in particular, may reduce the pressure felt on the user's ear when they are lying on their side. More particularly, embodiments in which the narrower neck portion 11 of the concave recess 6 is not completely compressed may reduce the pressure felt on the user's ear when they are lying on their side.

In a further embodiment of the present invention, a light filler material may be included in the recess 6 or cavity/void portion. Inclusion of a light filler material may assist with avoiding complete collapse of the recess 6 or cavity/void portion.

In embodiments where a recess 6 is provided in the upper surface 3 of the main body 1, the recess 6 may define a head receiving portion 8 in an upper surface of the head supporting portion 4. As a result, the part of the main body below the recess forms a region of increased deformability relative to other parts of the main body.

In embodiments where a cavity/void portion is provided adjacent the upper surface 3 of the main body 1, the head receiving portion 8 is formed by the weight of the user's head H compressing the cavity/void portion. As a result, the part of the main body 1 above the cavity/void portion forms a region of increased deformability relative to other parts of the main body. The recess 6 or cavity/void portion may be completely collapsed by the weight of the user's head, but more preferably is only partially or mostly compressed.

Embodiments of the present invention in which the recess 6 (or cavity/void portion) is provided in a lower surface 7 (or adjacent a lower surface 7) of the main body 1 may be more comfortable than pillows of the prior art in which a recess is formed in the upper surface of the pillow, as the transition between the head receiving portion 8 and the remainder of the head supporting portion 4 may be softened. This means that the user is not forced to place their head exactly in a predetermined head receiving portion.

Embodiments of the present invention in which the recess 6 (or cavity/void portion) is provided in an upper surface 3 (or adjacent an upper surface 3) of the main body 1 may be suitable for applications where the retention of the user's head in a substantially fixed position is desirable as the recess or cavity/void portion may tend to hold the user's head more fixedly when it is provided in (or adjacent) an upper surface 3 of the main body 1. In embodiments where a recess 6 is provided in the upper surface of the main body 1 the concave recess 6 may define a head receiving portion on an upper surface of the main body 1. For example, such embodiments of the present invention may be suitable for use in surgical or clinical settings or where the user suffers from a sleep disorder characterised by pauses in breathing or instances of shallow breathing during sleep such as sleep apnoea. In such embodiments it may be preferable to exclude the contour 16, described in further detail below.

In other embodiments a portion of the main body may be formed from or provided with a material that is more easily deformable than other parts of the main body so as to define a region of increased deformability. Such other material may, for example, be provided in the previously disclosed recess or cavity/void portion and/or part of the main body may be formed from such a more easily deformable material or provided otherwise by way of insert or attachment. As will be appreciated, the pillow may be integrally formed from one or more materials with the structural properties thereof varied as required.

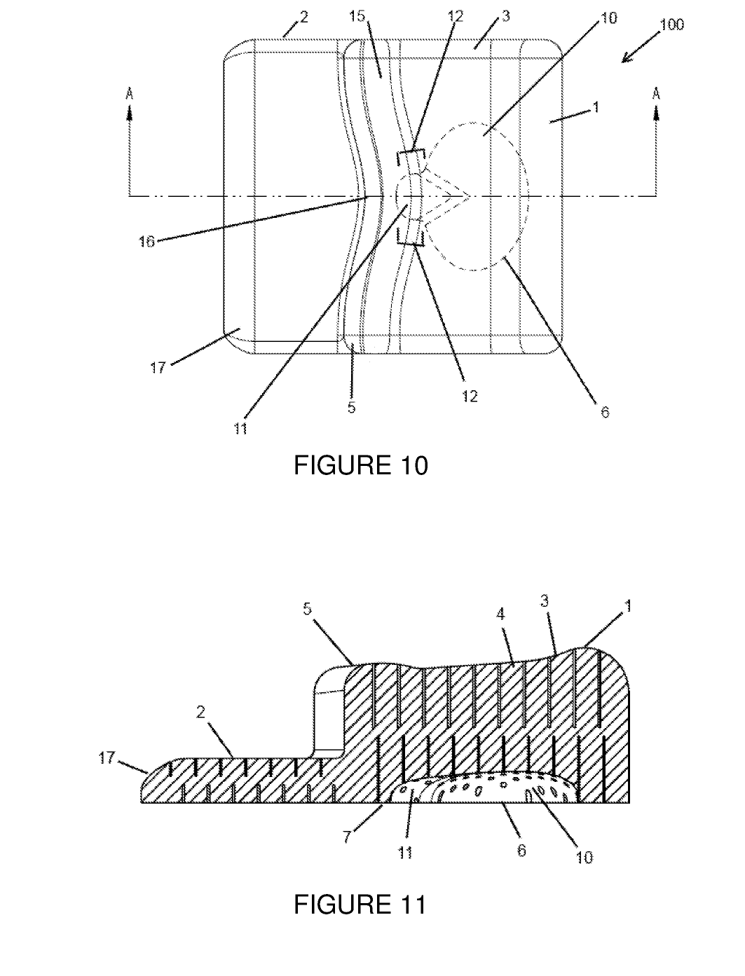

As is best seen in FIGS. 7 and 10, the recess 6 (or cavity/void portion) may comprise a wider portion 10 and a narrower neck portion 11. The narrower neck portion 11 is closer to the shoulder elevating or supporting portion 2 (if present, as is shown in FIGS. 6 to 12) than the wider portion 10. The wider portion 10 is substantially hemispherical in shape, but is elongated in the width direction of the pillow 100. The recess 6 (or cavity/void portion) is substantially bulb-shaped, having a wider bulbous head portion 10 and a narrower neck portion 11. The narrower neck portion 11 preferably slopes towards the lower surface of the pillow from the intersection of the narrower neck portion 11 with the wider portion 10.

The head supporting portion 6 is shaped to create a jaw support portion 12 on either side of the head receiving portion 8 in use. The jaw support portions 12 ensure that the user's jaw is held slightly higher than it would be if these portions were not present. In the pillows of the prior art the user's chin may tuck downward toward the chest when the user is lying on their side, thereby occluding their airway.

In other embodiments the head supporting portion is configured to the create jaw support portions on the head supporting portion substantially adjacent the lateral aspects of the head receiving portion on opposing sides of head receiving portion in use. To this end the region of increased deformability is configured to reduce the ease of deformability in the jaw support portions and/or the main body is configured to have increased resistance to deformation in or about the jaw support portions. For example, the main body may be thicker in these areas (at least relative to the region of increased deformability) and/or a less deformable material may be provided, either by provision of an insert or by varying the properties of the main body in different regions.

As is best seen in FIGS. 6, 7, 9 and 10 in certain embodiments the top edge and the shoulder-facing surface of the neck supporting portion define a contour having a medial concave portion and lateral convex portions on opposing sides. Preferably the midpoint of the medical concave portion of the contour is substantially aligned with the midpoint of the recess 6 and/or cavity/void portion. Preferably the medial concave portion of the contour is adapted to receive the user's shoulder when the user is lying on their side. This configuration assists in positioning the user's head relative to the recess 6 and/or cavity/void portion.

In certain embodiments the lateral convex portions of the contour are adapted to receive the user's jaw on the top shoulder-facing edge when the user is lying on their side such that the user's head is supported in a neutral position.

The medial concave portion of the contour preferably has a maximum depth of approximately 20 mm to 55 mm. For example, the medial concave portion of the contour may have a maximum depth of approximately 20 mm to 25 mm, approximately 25 mm to 30 mm, approximately 30 mm to 35 mm, approximately 35 mm to 40 mm, approximately 40 mm to 45 mm or approximately 45 mm to 50 mm. For example, the medial concave portion of the contour may have a maximum depth of approximately 25 mm to 50 mm or approximately 30 mm to 45 mm. Preferably the medial concave portion of the contour may have a maximum depth of approximately 30 mm to 40 mm, for example 35 mm.

A contour as described herein can also be provided in other embodiments of the invention.

In the embodiment shown in FIGS. 6 to 12 the neck supporting portion 5 comprises a ridge or lip 15, hereinafter referred to as a ridge. In preferred embodiments the ridge 15 is situated substantially on the upper surface of the neck supporting portion 5. More preferably the ridge 15 is situated substantially on the upper surface of the neck supporting portion and substantially adjacent the top edge of the shoulder-facing surface of the neck supporting portion 5.

In use, the ridge 15 assists in causing the user's head to be held substantially in line with the natural or neutral position of the user's head, and in particular when the user is lying on their side the ridge 15 assists in causing the users jaw to be slightly elevated relative to its position if the ridge 15 is absent. This configuration also assists in retaining the user's head on the pillow in use, providing added comfort to the user.

In one embodiment the ridge may be approximately 65 mm to 95 mm wide from the top edge of the shoulder-facing surface of the neck supporting portion toward the opposite edge of the main body. For example, the ridge may be approximately 65 mm to 70 mm wide, 70 mm to 75 mm wide, 75 mm to 80 mm wide, 80 mm to 85 mm wide, 85 mm to 90 mm or 90 mm to 95 mm wide. For example, the ridge may be approximately 70 mm to 90 mm wide, 75 mm to 85 mm wide, or 70 mm to 80 mm wide, for example 80 mm wide.

In one embodiment the ridge may have a height of approximately 5 mm to 15 mm.

In a particular embodiment of the present invention the ridge 15 and contour are adapted to act in concert to assist in causing the user's head to be held substantially in line with the natural or neutral position of the user's head, and in particular adapted to assist in causing the user's jaw to be slightly elevated when the user is lying on their side relative to its position if the configuration is absent, thus causing the user's jaw to be held in a neutral position. In such an embodiment, the ridge 15 substantially follows the shape of the contour.

In use, the user's head H and neck are held in a substantially neutral position. As seen in FIG. 10, the jaw support portions 12 combine with the head receiving portion 8 to cause the user's head to be held substantially in line with the natural or neutral position of the user's head. Therefore, when the user is lying on their side a central axis through the user's head, from neck to crown, is almost horizontal, but may slope upward very slightly. This opens the user's airways and reduces snoring when the user is lying on their side.

When on their back, the central axis is substantially horizontal, and an axis from the back of the user's head to the front of the user's head is substantially vertical. This straight and natural positioning of the user's head keeps airways open and reduces snoring.

In another embodiment (not shown), the pillow 100 may be wider than usual, and two (or more) concave recesses (or void spaces) may be provided side-by-side, so that the user may choose from a variety of positions, or, in the case of full width pillows, so that multiple users may use the pillow at the same time. In such an embodiment the pillow 100 may further comprise a plurality of contours having medial concave portions and lateral convex portions on opposing sides, in the manner of a sinusoidal wave form. Preferably the midpoint of each of the concave portions of the plurality of contours is substantially aligned with the midpoint of each of the plurality of concave recesses and/or void portions.

In other embodiments (not shown) the shoulder elevating or supporting portion 2 may be omitted, and the height of the upper surface of the pillow reduced slightly to compensate.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

Where in the foregoing description, reference has been made to specific components or integers of the invention having known equivalents, then such equivalents are herein incorporated as if individually set forth.

Although this invention has been described by way of example and with reference to possible embodiments thereof, it is to be understood that modifications or improvements may be made thereto without departing from the spirit or scope of the invention.

In particular, as will be appreciated, the dimensions described herein are exemplary only and have been provided in view of the stature and body weight of a typical user. The dimensions of the pillow or sleep positioner may be altered without departing from the spirit and scope of the invention, and the pillow or sleep positioner may be generally scaled up or down to accommodate a larger or smaller user, or simply based on user preferences. Furthermore, as will be appreciated, a tolerance may apply to dimensions described herein without departing from the spirit and scope of the invention.

Further, the dimensions of the pillow or sleep positioner may be varied depending on the properties of the material(s) forming it. Generally, the more compressible the material, the higher or greater depth required of the pillow or sleep positioner at rest, and vice versa, such that the user's head and neck are positioned comfortably but performing the required function of orienting the user's head during use.

1-26. (canceled)

27. A pillow comprising a main body, the main body comprising:
an upper surface and a lower surface; and
a neck supporting portion and a head supporting portion, wherein:
the main body comprises a region of increased deformability relative to other parts of the main body such that when a user's head is resting on the head supporting portion, a recess, preferably a concave recess, defines a head receiving portion in an upper surface of the head supporting portion;
the region of increased deformability is configured to create jaw support portions substantially adjacent the lateral aspects of the head receiving portion on opposing sides in use; and
the neck supporting portion comprises a ridge adapted to provide additional support to a user's jaw when the user is lying on their side such that the user's head is supported in a neutral position.

28. A pillow according to claim 27, further comprising a shoulder supporting portion.

29. A pillow according to claim 28, wherein the shoulder supporting portion is integrally formed with the main body.

30. A pillow according to claim 28, wherein the main body has increased thickness relative to the shoulder supporting portion.

31. A pillow according to claim 28, wherein the shoulder supporting portion has a thickness of between around 35 mm and 65 mm at rest and/or comprises latex and has a density of between approximately 40 kg/m³ and 100 kg/m³.

32. A pillow according to claim 27, wherein the region of increased deformability comprises a recess provided in the lower surface of the main body and/or a void portion and/or cavity provided inside the main body or adjacent a lower surface of the main body.

33. A pillow according to claim 27, wherein the region of increased deformability comprises a recess provided in the upper surface of the main body and/or a void portion and/or cavity provided inside the main body or adjacent an upper surface of the main body.

34. A pillow according to claim 27, wherein the recess and/or void portion and/or cavity is bulb-shaped, comprising a bulbous head portion and a narrower neck portion.

35. A pillow according to claim 34, wherein the bulbous head portion is approximately 1.05 times to 1.25 times longer along an elongated axis aligned generally perpendicular to the user's central axis in use and/or is approximately 225 mm to 255 mm along the elongated axis.

36. A pillow according to claim 27, wherein the recess and/or void portion and/or cavity has a maximum depth of substantially 25 mm to 55 mm at rest.

37. A pillow according to claim 27, wherein the head supporting portion is adapted to support a user's head in a substantially horizontal position when the user is lying on their back and/or is adapted to support a user's head in a neutral position when the user is lying on their side.

38. A pillow according to claim 27, wherein the neck supporting portion has a height of between 85 mm and 125 mm above an upper surface of the shoulder supporting portion when the shoulder supporting portion is present, or between 85 mm and 125 mm above the surface the user lies on when the shoulder supporting portion is not present.

39. A pillow according to claim 27, wherein the ridge is approximately 65 mm to 95 mm wide from the top edge of the shoulder-facing surface of the neck supporting portion toward the opposite edge of the main body and/or has a height of approximately 5 mm to 15 mm.

40. A pillow according to claim 34, wherein the narrower neck portion is proximate to the neck supporting portion such that an end of the narrower neck portion distal from the bulbous head portion terminates in a region directly below said ridge.

41. A pillow according to claim 27, wherein a top edge and a shoulder-facing surface of the neck supporting portion define a contour having a medial concave portion and lateral convex portions on opposing sides.

42. A pillow according to claim 41, wherein the medial concave portion of the contour is adapted to receive a user's shoulder when the user is lying on their side and/or has a maximum depth of approximately 20 mm to 5 mm at rest.

43. A pillow according to claim 41, wherein the lateral convex portions of the contour are adapted to receive a user's jaw on the top shoulder-facing edge when the user is lying on their side, such that the user's head is supported in a neutral position.

44. A pillow according to claim 27, wherein the main body comprises latex and has a density of between approximately 40 kg/m³ and 100 kg/m³.

45. A pillow according to claim 27, wherein the head supporting portion has a slight incline from a first end region to a second end region, away from the neck supporting portion, such that the second end portion of the head supporting portion is higher relative to the first end portion.

46. A pillow comprising a main body, the main body comprising:
an upper surface and a lower surface;
a neck supporting portion and a head supporting portion; and
a shoulder supporting portion; wherein:
the main body comprises a region of increased deformability relative to other parts of the main body such that when a user's head is resting on the head supporting portion, a recess, preferably a concave recess, defines a head receiving portion in an upper surface of the head supporting portion;
the region of increased deformability is configured to create jaw support portions substantially adjacent the lateral aspects of the head receiving portion on opposing sides in use;
the neck supporting portion comprises a ridge adapted to provide additional support to a user's jaw when the user is lying on their side such that the user's head is supported in a neutral position; and
a top edge and a shoulder-facing surface of the neck supporting portion define a contour having a medial concave portion and lateral convex portions on opposing sides.
